(19) United States
(12) Patent Application Publication
(10) Pub. No.: US 2017/0354431 A1
(43) Pub. Date: Dec. 14, 2017

(54) SURGICAL INSTRUMENT WITH INTERNAL IRRIGATION

(71) Applicant: Medtronic Xomed, Inc., Jacksonville, FL (US)

(72) Inventors: Joshua David Rubin, Jacksonville, FL (US); Jeffrey A. Bowman, Jacksonville, FL (US); David C. Fowler, Jacksonville, FL (US)

(73) Assignee: Medtronic Xomed, Inc., Jacksonville, FL (US)

(21) Appl. No.: 15/687,684

(22) Filed: Aug. 28, 2017

Related U.S. Application Data
(63) Continuation of application No. 12/109,713, filed on Apr. 25, 2008, now abandoned.

(51) Int. Cl. A61B 17/32; A61M 1/00; A61B 17/24

(52) U.S. Cl. CPC A61B 17/32002; A61M 1/0082; A61B 17/24; A61B 2217/007; A61B 2017/320004; A61B 2217/005

(57) ABSTRACT

A surgical cutting instrument comprises an outer tubular member and an inner tubular member. The outer tubular member includes a proximal section, an intermediate section, a distal section, and a side wall. The side wall defines a central lumen extending from the proximal section to the distal section. The side wall also includes means for conducting fluid within a sidewall of the outer tubular member from the proximal section to the distal section and includes a distal opening positioned to direct fluid at the bur and a treatment site. The inner tubular member is rotatably received within the central lumen. A distal end of the inner tubular member forms a bur extending distally beyond, and exposed relative to, the distal section of the outer tubular member.

### CROSS REFERENCE

This Application is a Continuation of U.S. patent application Ser. No. 12/109,713, filed Apr. 25, 2008, entitled SURGICAL INSTRUMENT WITH INTERNAL IRRIGATION, and is incorporated herein by reference.

### BACKGROUND

Powered surgical instruments have been developed for use in many ear-nose-throat (ENT) operations as well as other operations in and around the skull. One type of cutting instrument includes a bur supported by an inner tubular member that is rotatable with respect to an outer tubular member. The bur is used to debride a target tissue of a treatment site. In many instances, the bur and/or treatment site are irrigated to facilitate lubrication of the treatment site as well as to cool the bur. In other instances, aspiration is applied to the treatment site to remove debrided tissue as well as to remove excess fluid. However, conventional cutting instruments that include an aspiration mechanism and/or an irrigation mechanism do so by externally attaching an aspiration tube or an irrigation tube that extends along a length of an outer surface of the cutting instrument. While the additional functions of aspiration and irrigation are gained, this added functionality comes at a high price because these external aspiration/irrigation tubes substantially increase a cross-sectional profile of the cutting instrument. This increased cross-sectional profile can reduce the number and/or type of treatment sites accessible by the conventional cutting instrument. Moreover, a distal end of these external aspiration/irrigation tubes increase the likelihood of the cutting instrument catching on soft tissues and bony structures encountered along the entry pathway of the cutting instrument to the treatment site.

Accordingly, conventional surgical instruments including external irrigation pathways can reduce the effectiveness of micro-burring instruments by hampering access through narrow entryways and into small treatment sites.

### BRIEF DESCRIPTION OF THE DRAWINGS

FIG. 1 is perspective view of a system including a surgical debriding instrument, in accordance with principles of the present disclosure;

FIG. 2 is as assembly view of the instrument, in accordance with principles of the present disclosure;

FIG. 3 is an enlarged partial cross-sectional view of the instrument of FIG. 2;

FIG. 4 is a schematic illustration of irrigating a treatment site using a debriding instrument, in accordance with principles of the present disclosure.

FIG. 5 is a top plan view of an outer portion of an outer tubular member of a debriding instrument, in accordance with principles of the present disclosure;

FIG. 6 is a cross-sectional view of the instrument as taken along lines 6-6 of FIG. 5;

FIG. 7 is an enlarged partial cross-sectional view of a proximal portion of the instrument of FIG. 5 as secured within an outer hub, in accordance with principles of the present disclosure;

FIG. 8 is a top plan view of an inner portion of the outer tubular member of the debriding instrument, in accordance with principles of the present disclosure;

FIG. 9 is a cross-sectional view of the instrument as taken along lines 9-9 of FIG. 8;

FIG. 10 is a perspective view of the outer tubular member illustrating the interior passages of the side wall of the outer tubular member, in accordance with principles of the present disclosure;

FIG. 11 is a cross-sectional view of the outer tubular member as taken along lines 11-11 of FIG. 10.

FIG. 12 is a top plan view of an instrument including an angled distal portion, in accordance with principles of the present disclosure;

FIG. 13 is a perspective view of an instrument and a handpiece, in accordance with principles of the present disclosure;

FIG. 14 is a side plan view of an instrument, in accordance with principles of the present disclosure; and

FIG. 15 is schematic illustration of irrigating a treatment site using a debriding instrument including an internal aspiration pathway, in accordance with principles of the present disclosure.

### DETAILED DESCRIPTION

Embodiments of the present disclosure are directed to cutting instruments having a low cross-sectional profile to enable their application in smaller treatment sites and/or to facilitate their access to a treatment site through narrow passageways.

In one embodiment, the cutting instrument includes an inner tubular member rotatably received within an outer tubular member and which includes a bur at its distal end. The inner tubular member and the outer tubular member each include a hub to facilitate their rotational relationship and their control by a handpiece that further supports both the inner tubular member and the outer tubular member. Rotation of the bur via rotation of inner tubular member causes debriding of the target tissue at a treatment site.

The outer tubular member includes a side wall defining an interior passage that acts as an irrigation pathway to supply an irrigation fluid to the treatment site adjacent to the bur. Because the irrigation pathway is incorporated internally and not provided through an external tube (as in conventional cutting instruments), the cutting instrument has a low cross-sectional profile. This smaller cross-sectional profile enables insertion of distal cutting end of the instrument into smaller treatment sites and facilitates introduction of the distal cutting end through narrow and/or curved passageways that provide access to the treatment site. In another aspect, by providing the irrigation pathway within a sidewall of the outer tubular member, interaction of the irrigation fluid with the inner tubular member (or other components internal to cutting instrument) is avoided.

In some embodiments, the bur and the inner tubular member further define an aspiration pathway through an interior of the bur (and the inner tubular member) to avoid the conventional arrangement of an external aspiration tube of the types typically used in conventional instruments. In the embodiments, the inner tubular member has a length so that the aspiration pathway may extend continuously through a hub assembly of both the inner tubular member and the outer tubular member. Accordingly, with this arrangement, the internally incorporated aspiration pathway further maintains the low cross-sectional profile that is achieved via arranging the irrigation pathway within a side wall of the outer tubular member, as described above.

Surgical instruments embodying principles of the present disclosure can be employed in various types of surgery including, but not limited to, various sinus procedures, skull base tumor removal (such as pituitary tumors, clivus chordomas, etc.), mastoidectomy, temporal bone tumor removal, craniotomy, a modified Lothrop procedure, spinal diseases, and the like.

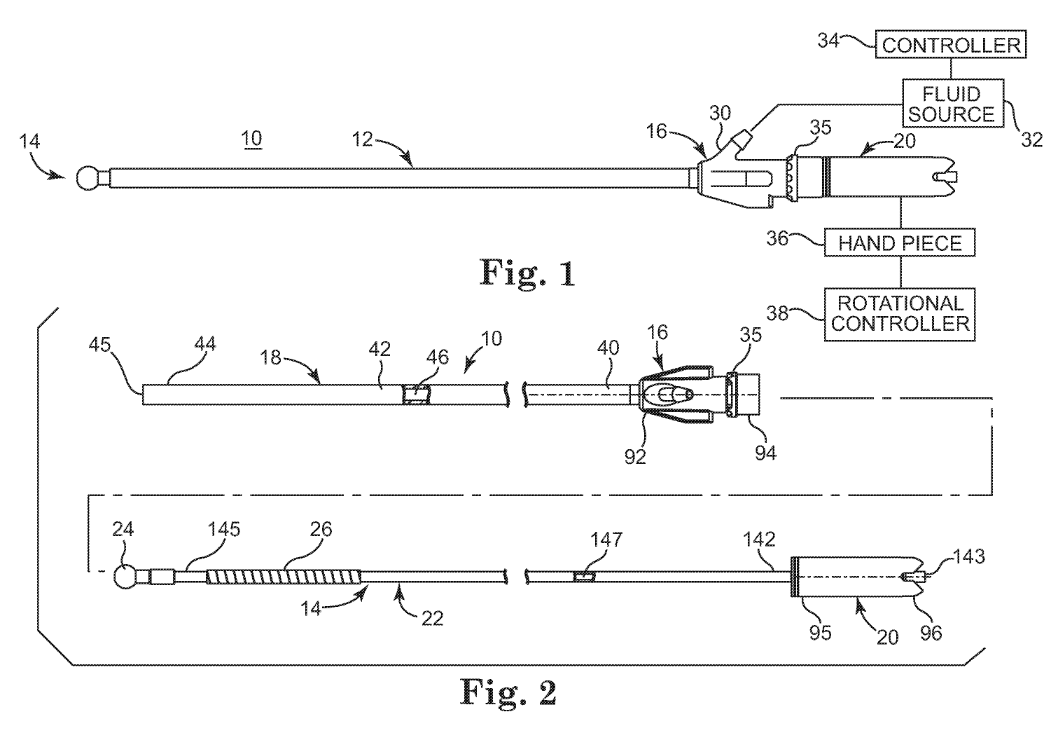

These and other embodiments are described more fully in association with FIGS. 1-15.

One preferred embodiment of a surgical micro-burring instrument 10 is illustrated in FIGS. 1-2. The instrument 10 includes an outer tubular assembly 12 and an inner tubular assembly 14 (referenced generally in FIG. 1). With particular reference to FIG. 2, the outer tubular assembly 12 includes an outer hub 16 and an outer tubular member 18, whereas the inner tubular assembly 14 includes an inner hub 20 and an inner tubular member 22. The inner tubular member 22 is sized to be coaxially received within the outer tubular member 18 and forms a bur 24. The inner tubular member 22 includes a proximal section 142 with end 143 and a distal section 145. In some embodiments, inner tubular member 22 additionally comprises a spring section 26 positioned proximal to bur 24 at distal section 145. In one aspect, an inner surface of inner tubular member 22 defines a lumen 147. As described in greater detail below, the micro-burring instrument 10 is configured to optimally perform a surgical procedure, such as a sinus procedure or one of the other procedures noted above.

As illustrated in FIG. 1, the outer tubular member 18 extends distally from the outer hub 16. To this end, the outer hub 16 can assume a wide variety of forms known in the art. In some embodiments, outer hub 16 comprises an irrigation port 30 configured for fluid communication via tubing (not shown) with a fluid source 32 controlled by controller 34.

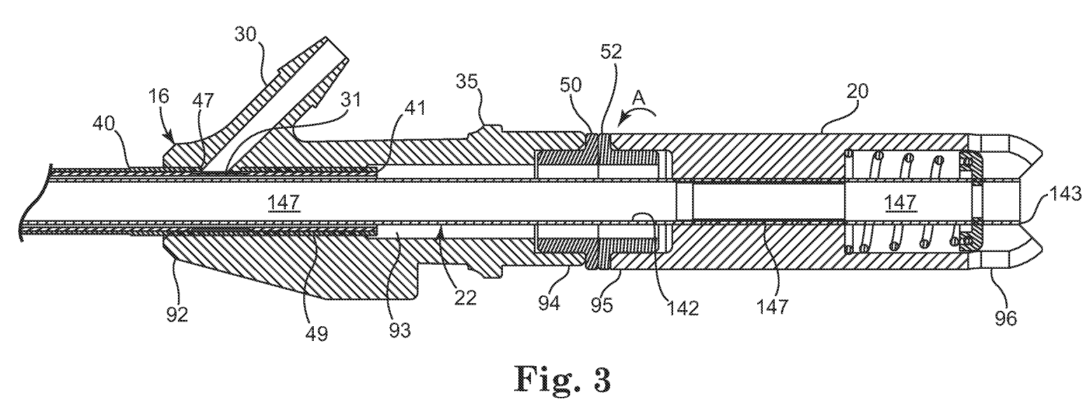

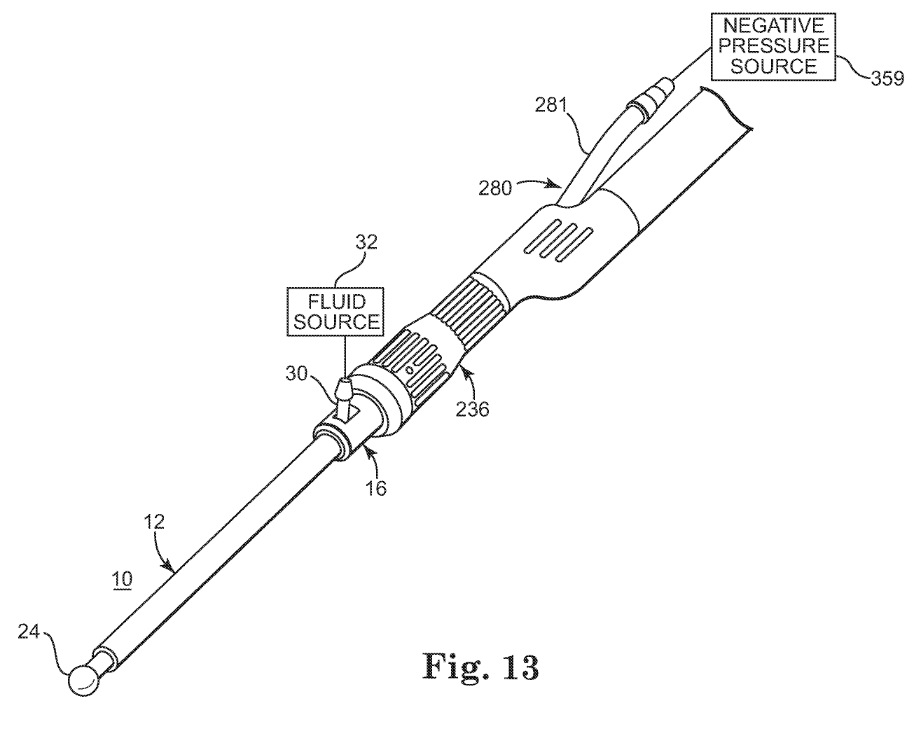

As illustrated in FIG. 1 and with additional reference to FIG. 3, the inner tubular member 22 extends distally from inner hub 20. With continued reference to FIG. 1, in some embodiments, inner hub 20 is configured to be engaged by a handpiece 36 (or handpiece 236 in FIG. 13) for handling instrument 10. In particular, rotational controller 38 (via a connection between handpiece 36 and inner hub 20) enables selective rotational control over inner tubular member 22 to cause high-speed rotation of bur 24 for debriding or otherwise cutting a target tissue.

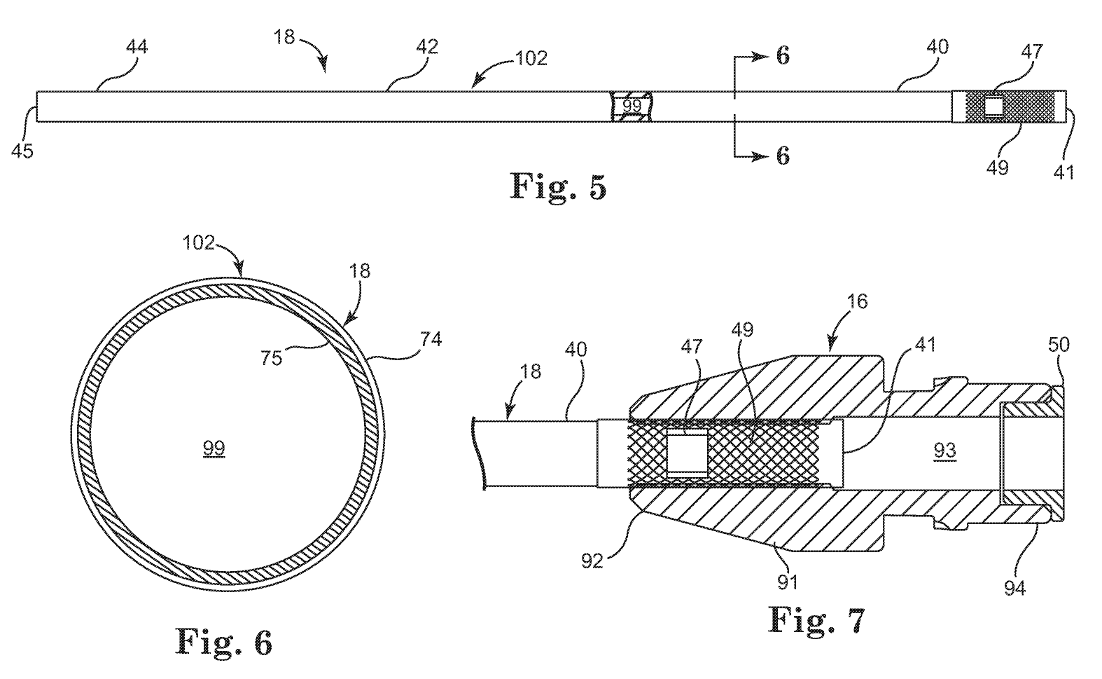

With reference to FIG. 2, the outer tubular member 18 is an elongated tubular body defining a proximal section 40 with proximal end 41 (FIG. 5), an intermediate section 42, a distal section 44 with distal end 45 (FIG. 5), and a central lumen 46. The central lumen 46 extends from the proximal section 40 to the distal section 44. In this regard, and as described in greater detail below, the distal section 44 is open at a distal end 45 thereof to enable the inner tubular member 22 to extend distally beyond the distal end 45 of outer tubular member 18. Similarly, the proximal section 40 is open at a proximal end 41 thereof to facilitate positioning of the inner tubular member 22 within the central lumen 46. Moreover, with additional reference to FIGS. 3, 5, and 7, proximal section 40 comprises a proximal window 47 located distally of proximal end 41. In some embodiments, proximal section 40 additionally comprises a knurled portion 49 located on a surface of proximal section 40 and that surrounds the proximal window 47. In one aspect, knurled portion 49 facilitates securing proximal section 40 to an inner portion of outer hub 16, as illustrated in FIGS. 3 and 7.

In one suitable configuration, as illustrated in FIG. 7, the proximal portion 40 is inserted into a lumen 93 of outer hub 16 to secure knurled portion 49 within the distal section 92 and intermediate section 91 of outer hub 16. While better seen in FIG. 3, the proximal section 40 is advanced proximally within lumen 93 of outer hub 16 until window 47 is aligned underneath a bottom opening 31 of irrigation port 30, and then secured in this position to maintain fluid communication between irrigation port 30 and proximal window 47. In addition, in this configuration, proximal end 41 is open to lumen 93 of outer hub 16. Accordingly, in one aspect, the proximal section 40 has an outer diameter adapted to receive the outer hub 16 thereon.

However, the remainder of the outer tubular member 18 preferably provides a relatively uniform outer diameter (as represented by reference numeral 74 in FIG. 6) selected to perform the desired sinus procedure and a relatively uniform inner diameter (as represented by reference numeral 75 in FIG. 6) selected to rotatably receive the inner tubular member 22. For example, in one embodiment, the intermediate section 42, as well as the distal section 44 to permit use of the inner tubular member 22/burr 24 as part of a sinus procedure.

Returning to FIG. 2, the inner tubular member 22 extends from the inner hub 20. In one preferred embodiment, the inner hub 20 is configured for selective attachment to handpiece 36 (and as also described in association with FIG. 13) that can be operated to automatically rotate the inner tubular member 22 during use.

As previously described, the inner tubular member 22 forms bur 24 at a distal end thereof. In general terms, bur 24 is a solid member that can assume a variety of forms and is adapted with an abrasive or rough surface to cut or abrade bodily tissue upon rotation thereof. In some embodiments, the bur 24 forms a cutting surface including one or more cutting elements. While a spherical bur configuration is shown, it will be appreciated that other configurations can be used including, but not limited to, cylindrical, hemispherical, ellipsoidal, and pear-shaped configurations.

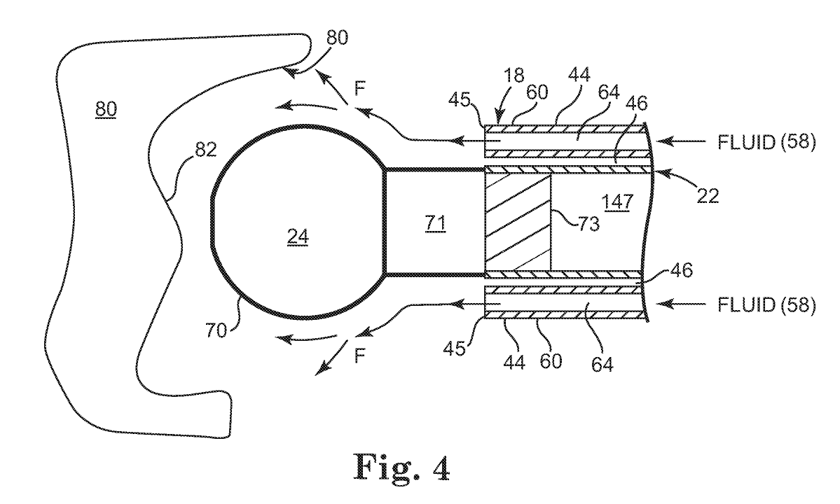

With reference to FIGS. 1-3, the micro-burring instrument 10 is assembled by coaxially positioning the inner tubular member 22 within the outer tubular member 18 via the central lumen 46. With particular reference to FIG. 3, a seal portion 52 of the inner hub 20 (at distal end 95 of inner hub 20) abuts against a seal portion 50 of the outer hub 16. With this in mind, the inner tubular member 22 and inner hub 20 of inner assembly 14 is rotatable relative to the outer tubular member 18 and outer hub 16 of outer assembly 12. To this end, a distance of separation between the inner hub 20 and the bur 24 is greater than a distance of separation between the outer hub 16 and the distal end 45 of outer tubular member 18, thereby dictating that a desired position of the bur 24 will be exposed relative to the outer tubular member 18, as best shown in FIGS. 1 and 4. In particular, the inner tubular member 22 is coaxially disposed within the outer tubular member 18 such that the distal end 45 of the outer tubular member 18 is proximal to the bur 24 and to the distal end 145 of inner tubular member 22.

As illustrated by FIGS. 1-2 and with additional reference to FIG. 4, once bur 24 is positioned at treatment site 80 to debride target tissue 82, fluid 58 supplied from fluid source 32 flows through an interior passage 64 of side wall 60 of outer tubular member 18 to irrigate bur 24 and/or the treatment site 80. In one aspect, this arrangement enables flooding the treatment site 80 with fluid 58 (and as further represented by arrows F), as appropriate to the procedure, while the bur 24 is rotating to cut the target tissue 82. In some embodiments, the fluid 58 irrigates the treatment site 80 before and/or after the bur 24 rotates to cut the target tissue 82. While side wall 60 can take many forms, one particular embodiment is illustrated in FIGS. 6-12, as described in more detail hereafter.

With further reference to FIG. 4, bur 24 includes a shaft 71 extending distally from (and secured relative to) distal section 44 of inner tubular member 22 and a tip 70 shaped to debride the target tissue 82. In one aspect, proximal end 73 of bur 24 blocks lumen 147 of inner tubular member 22 to prevent any fluid or other substances from entering lumen 147 near bur 24. Moreover, while tip 70 is shown as having a generally spherical shape in FIG. 4, bur 24 can take other forms, as previously described in association with FIGS. 1-2.

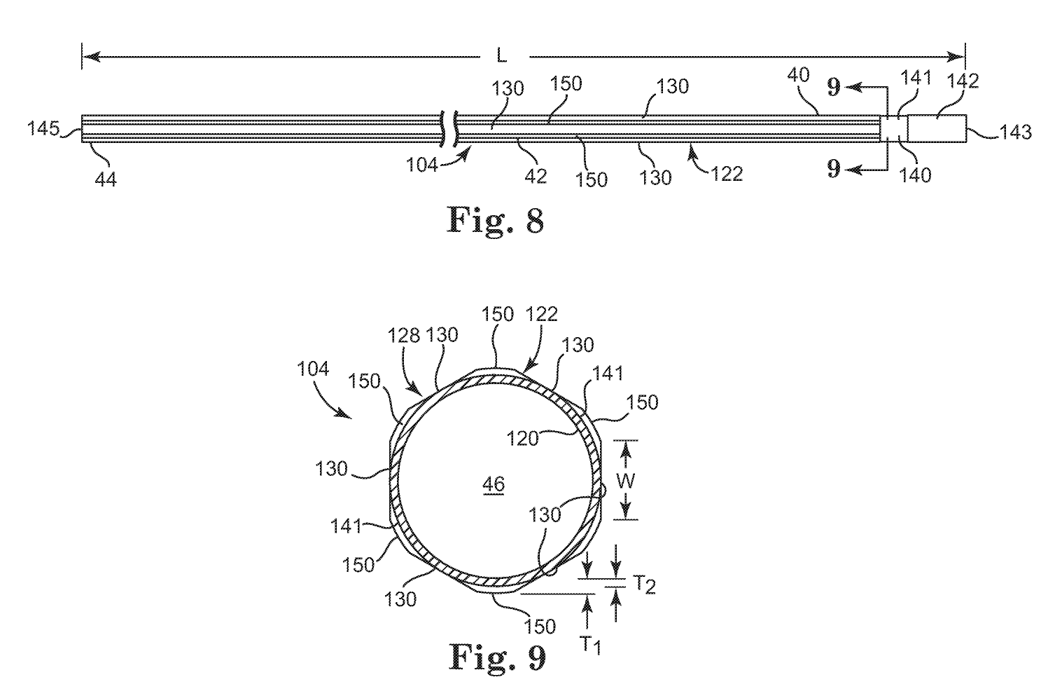

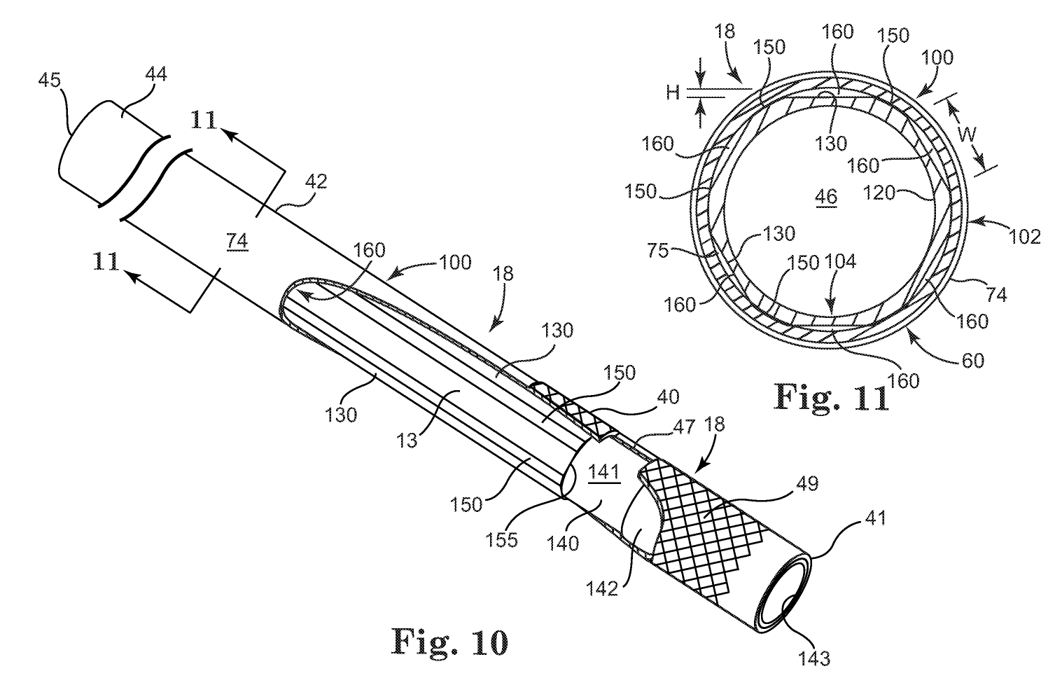

While outer tubular member 18 was previously described in association with FIGS. 1-2, outer tubular member 18 can take many forms to achieve the configuration of a side wall 60 that defines an interior passageway 64 configured to provide fluid to cool bur 24 and/or lubricate treatment site 80, as previously described in association with FIG. 4. Nevertheless, in one configuration, outer tubular member 18 comprises an assembly 100 formed from an outer portion 102 shown in FIGS. 5-7 and an inner portion 104, as shown in FIGS. 8-9. Outer portion 102 and inner portion 104 comprise two separate members that are joined together to produce an assembly 100 having the form shown in FIGS. 10-11. For the sake of illustrative clarity, each of the inner portion 102 and the outer portion 104 will be further described separately.

FIG. 6 is a cross-sectional view of outer portion 102 of outer tubular member 18 and illustrates outer portion 102 defining a hollow sleeve. In one aspect, an outer surface of outer portion 102 of outer tubular member 18 comprises substantially the same features and attributes that were previously described in association with FIGS. 3, 5, and 7 for outer tubular member 18 as a whole. In one aspect, FIG. 6 further illustrates outer portion 102 including an inner surface 75 that defines a diameter sized and adapted to receive inner portion 104. Outer portion 102 also defines an outer surface 74 which forms the outer surface of outer tubular member 18 and which provides a generally uniform and generally smooth outer diameter.

FIG. 8 is a side plan view of inner portion 104 of outer tubular member 18 and FIG. 9 is a cross-sectional view of inner portion 104, according to principles of the present disclosure. While inner portion 104 can take many forms, in the one configuration shown in FIGS. 8-9, inner portion 104 defines an inner surface 120 and an outer surface 122. The inner surface 120 defines a generally uniform diameter and is generally uniformly smooth from the proximal section 40, through the intermediate section 42, to the distal section 44. However, the outer surface 122 defines an array 128 of elongate recesses 130 extending from the distal section 44, along intermediate section 42, and through at least a portion of proximal section 40. In one embodiment, the elongate recesses 130 extend along a majority of the length of inner portion 104 (and therefore a majority of a length of outer tubular member 18) before terminating adjacent a circular recess 140 that extends transversely to the elongate recesses 130. In one aspect, circular recess 140 forms a ring extending about a circumference of outer surface of inner portion 104. The circular recess 140 is in fluid communication simultaneously with each of the elongate recesses, as will be further illustrated later in FIG. 10.

As illustrated in FIG. 8, in one aspect, outer surface 122 of inner portion 104 further defines a non-recess portion 142 proximal to circular recess 140. This non-recess portion 142 is sized and adapted to be sealingly secured to an inner surface 75 of outer portion 102. In one embodiment, non-recess portion 142 is laser welded relative to inner surface 75 of outer portion 102. This arrangement secures the inner portion 104 to outer portion 102 at proximal section 40 of outer tubular member 18 (located proximal to proximal window 47 shown in FIGS. 5 and 7) while simultaneously defining a terminal end of the fluid communication pathway that extends generally within sidewall 60 of outer tubular member 18. Accordingly, fluid flowing into outer tubular member 18 at proximal section 40 (from port 30 and fluid source 32) will enter through proximal window 47 of outer tubular member 18, and flow through circular recess 130 (FIGS. 3, 5, and 7) just distal to non-recess portion 142 of inner portion 104 before proceeding into recesses 130.

As best seen in FIG. 9, the elongate recesses 130 of inner portion 104 (of outer tubular member 18) form an array 128 of recesses 130 uniformly spaced apart about the circumference of inner portion 104 with each elongate recess 130 being defined between an adjacent pair of raised protrusions 150 formed on outer surface 122 of inner portion 104. In the one configuration shown in FIG. 9, array 128 includes six elongate recesses 130 that are spaced apart uniformly (i.e., equidistant from each other) about the circumference of outer surface 122 of inner portion 104. Of course, in other configurations, there can be greater or fewer than six elongate recesses 130. Nevertheless, at least one recess 130 is provided to form interior passageway 64 in side wall 60 of outer tubular member 18. Configurations with a greater number of recesses (instead of fewer recesses) spaced apart uniformly about the circumference of the inner portion (and consequently about the circumference of the outer tubular member 18) provide more balance to the fluid flow through side wall 60. This arrangement enables outer tubular member 18 to have a smaller thickness of the side wall because each recess 130 can have a smaller thickness or height (as represented by H in FIG. 11) while enabling generally the same volume of fluid to flow within the side wall 60 of the outer tubular member 18.

While a variety of techniques may be used to form the inner portion 104, in one embodiment inner portion 104 is formed by providing a generally tubular sleeve (not shown) having a first thickness and then cutting an outer surface of the sleeve (corresponding to outer surface 122) to create each elongate recess 130. Accordingly, with reference to FIG. 9, the protrusions 150 generally define the original, first thickness (as represented by T1) of the sleeve while the recesses 130 extending between the respective protrusions 150 comprise a second thickness (as represented by T2) substantially less than the first thickness. The difference between the first thickness and the second thickness will then define a height of the recess 130, as best seen in FIG. 11. In one aspect, the height of each recess 130 (as represented by H, the difference between T1 and T2), the width of each recess 130 (as represented by W), and the number of recesses defines the cross-sectional area available to send fluid through the interior passageway 64 within the sidewall 60 of outer tubular member 18.

FIG. 10 is a perspective view of assembly 100 of outer tubular member 18 showing inner portion 104 and outer portion 102 in an assembled state to form outer tubular member 18. FIG. 11 is cross-sectional view of assembly 100 of FIG. 10 that further illustrates the relationship between inner portion 104 and outer portion 102 of assembly 100 of outer tubular member 18.

As seen in FIGS. 10-11, after slidably inserting inner portion 104 within outer portion 102, inner portion 104 becomes coaxially disposed within outer portion 102. With this arrangement, the protrusions 150 contact inner surface 75 of outer portion 102, thereby forming separate conduits 160 between each of the elongate recesses 130 and inner surface 75 of outer portion 102. Accordingly, in one aspect, each adjacent pair of protrusions 150 defines the side walls of each respective conduit 160. The conduits 160 extend a majority of a length (represented by L1 in FIG. 8) of the outer tubular member 18 to provide a fluid communication pathway from a proximal section 40 (at which fluid 58 is supplied from irrigation port 30 via proximal window 47 (FIG. 5) and via circular recess 140) to the distal section 44. In one aspect, a surface 141 of circular recess 140 (also seen in FIG. 9) and a bottom portion of each recess 130 have substantially the same elevation at junction 155 (between circular recess 150 and the respective recesses 130) to provide a generally seamless transition therebetween.

Accordingly, one or more conduits 160 shown in FIGS. 10-11 correspond to (and define just one configuration of) interior passage 64 of side wall 60 of outer tubular member 18 that was previously described in association with FIG. 4. Therefore, conduits 160 define a fluid flow pathway internally within side wall 60 of outer tubular member 18 to deliver fluid 58 (from fluid source 32) to bur 24 and target tissue 82 at treatment site 80. As previously noted, this delivered fluid will flood the treatment site 80 to cool the bur 24 during rotation and/or to lubricate the target tissue 82, thereby increasing the effectiveness of the debriding action of the bur 24.

Moreover, because the irrigation fluid pathway is contained internally within the sidewall 60 of the outer tubular member 18, the outer tubular member 18 has a smaller overall cross-sectional profile. In another aspect, the outer surface 74 of the outer tubular member 18 is generally uniform and generally smooth without significant protrusions, such as the protrusion(s) that would otherwise be formed by an irrigation tube externally attached to instrument as seen in conventional instruments. With this in mind, this smaller cross-sectional profile provides instrument 10 with greater maneuverability to enable distal section 44 of instrument 10 to pass through various soft tissues and bony structures with less likelihood of the instrument 10 catching on soft tissues and bony structures encountered along a path to a treatment site at which rotation of bur 24 is deployed.

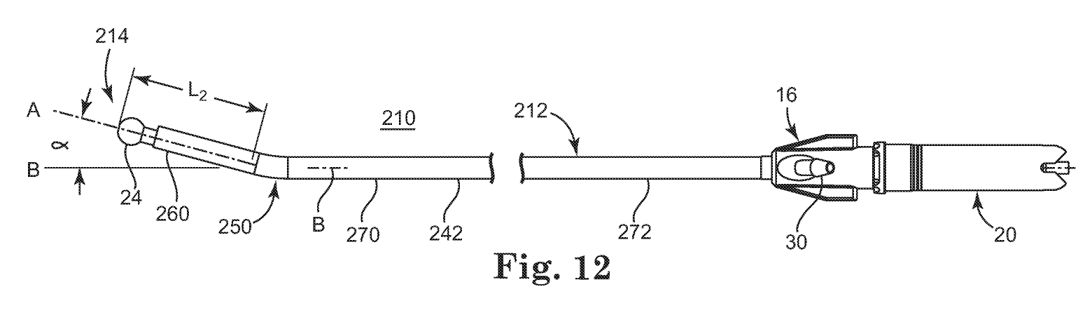

While the micro-burring instrument 10 of the present disclosure has been illustrated as being relatively straight (e.g., relative to the view of FIG. 1, the outer tubular member 18 is relatively straight), other configurations can be employed to facilitate a desired procedure. For example, FIG. 12 illustrates an alternative embodiment micro-burring instrument 210 highly useful for a sinus procedure that again includes an outer tubular assembly 212 and an inner tubular assembly 214 (illustrated generally). The outer and inner tubular assemblies 212, 214 comprise, in one embodiment, substantially the same features and attributes as the outer and inner tubular assemblies 12, 14 (respectively) as previously described in association with FIGS. 1-11. However, with the alternative embodiment instrument 210 of FIG. 12, the outer and inner tubular members 212, 214 define a slight bend, as referenced generally by 250, at a junction between a distal end portion 260 and an intermediate portion 270 of the instrument 210. In one embodiment, the bend 250 is configured to cause the a central axis (as represented by dashed line A) of the distal end portion 260 to define an angle a in the range of 10°-70°, relative to a central axis (as represented by dashed line B) of the intermediate portion 270 and proximal portion 272 of the instrument 210. Among other uses, this bend is particularly useful in properly positioning the distal end portion 260 during a skull-based procedure, among other surgical procedures favoring a bend 250 in distal end portion 260. To facilitate necessary rotation of the inner tubular assembly 214 in the region of the bend 250 (such as for rotating the bur 24 at a distal end thereof), an inner tubular member (hidden in the view of FIG. 12, but akin to the inner tubular member 22 of FIG. 2) is preferably flexible and formed of an appropriate material such as spiral wrap technology. Alternatively, other constructions can be employed.

Regardless of exact form, the micro-burring instrument 10, 210, of the present invention is useful in performing various sinus operations and other procedures. By way of example, and with reference to the one embodiment of FIGS. 1 and 2, the assembled instrument 10 is deployed to the target site. For example, in a surgical procedure, the instrument 10 is maneuvered to the treatment site 80 and the bur 24 is positioned against the bone or other target tissue 82, as illustrated in FIG. 4. Other related surgical techniques may be performed before, during, or after application of instrument 10.

Next, the inner tubular member 22 is then rotated relative to the outer tubular member 18, such that the bur 24 burs (e.g., cuts or abrades) the contacted cartilage and/or bone. As best seen in FIG. 4, the bur 24, and thus the target site 82, are periodically or continuously flushed with an irrigation fluid via the interior passage 64 (for example, the irrigation conduits 160) extending within the side wall 60 of the outer tubular member 18.

In addition to the surgical procedure described above, the micro-burring instrument 10, 210 of the present disclosure can be used to perform a variety of other surgical procedures in which hard tissue is debrided or cut while flooding the treatment site with fluid to irrigate the bur and the target tissue.

In one embodiment, the micro-burring instrument 10, 210, is attached to a powered handpiece 236 as shown in FIG. 13. The handpiece 236 can assume a variety of forms known in the art, and in one preferred embodiment comprises a StraightShot® powered handpiece, marketed by Medtronic-Xomed. In some embodiments in which a surgical instrument supports aspiration, and as illustrated in FIG. 13, handpiece 236 supports aspiration tubing 281 which forms part of an aspiration pathway 280 that extends distally through an interior of handpiece 236 (for fluid communication with an aspiration lumen associated with the instrument) and which extends proximally to be in fluid communication with negative pressure source 359.

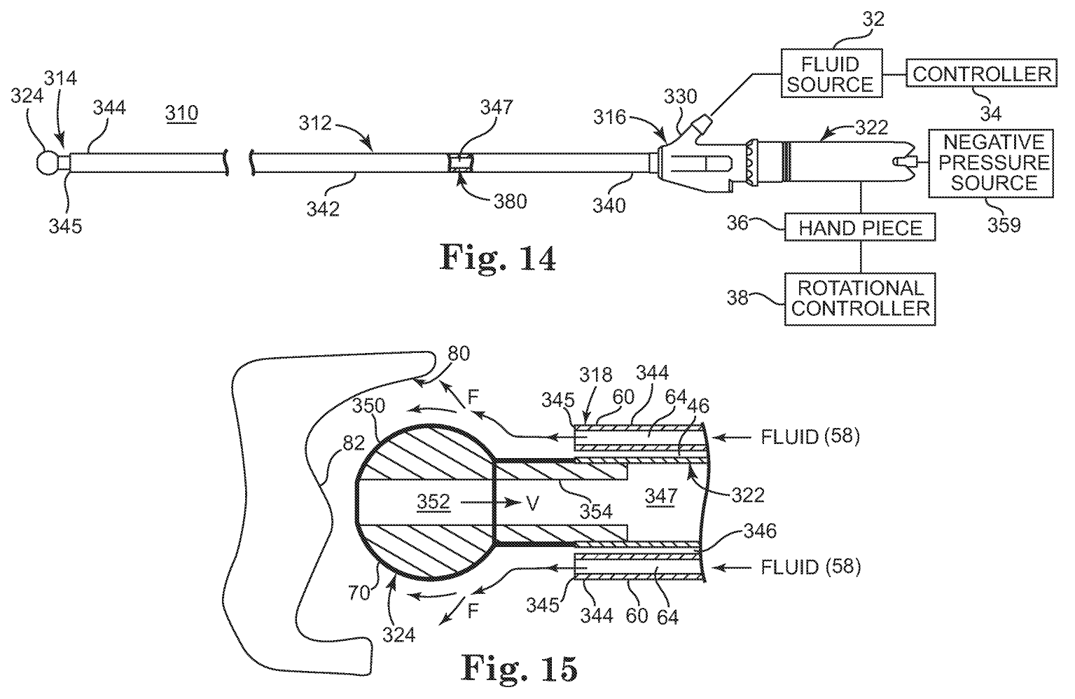

In one particular embodiment, instrument 10 takes a modified form as an instrument 310 illustrated and described in association with FIGS. 14-15. For example, FIGS. 14-15 illustrate another alternative embodiment micro-burring instrument 310 highly useful for a surgical procedure that again includes an outer tubular assembly 312 and an inner tubular assembly 314 (illustrated generally). The outer and inner tubular assemblies 312, 314 include, in one embodiment, substantially the same features and attributes as the outer and inner tubular assemblies 12, 14 (and 212, 214) previously described in association with FIGS. 1-13. However, with the alternative embodiment instrument 310 of FIGS. 14-15, the inner tubular assembly 314 defines an aspiration pathway 380 extending through a central lumen 347 of an inner tubular member 322 and inner hub 320 for connection to and fluid communication with negative pressure source 359 (via handpiece 36 or 236).

With additional reference to FIG. 15, a distal end 350 of the bur 324 forms a conduit 352 that extends through shaft 354 of bur 324 and which is open to the central lumen 347 defined by inner tubular member 322. By forming conduit 352 to extend through bur 324, a smaller overall, cross-sectional profile of instrument 310 is maintained in accordance with the smaller cross-sectional profile achieved via providing an irrigation pathway 280 within interior passage 64 (for example, the conduits 160 of FIGS. 10-11) of side wall 60 of outer tubular member 318. Regardless, the central lumen 347 serves as an aspiration conduit for the micro-burring instrument 310 (FIG. 1). Further, with reference to FIG. 15, when instrument 310 including aspiration pathway 280 including central lumen 347 is applied to treat a target site 82 (FIG. 4) the conduit 352 extending through bur 324 enables periodic or continuous aspiration (as represented by arrow V) via the central lumen 347 of the inner tubular member 22 to remove abraded tissue from the target site 82.

Nevertheless, it is understood that an alternative embodiment can be formed by modifying the embodiment of instrument 10 (FIGS. 1-12) to include an exteriorly extending aspiration passage proximal the bur 24 that is otherwise fluidly connected to the central lumen 147. This arrangement provides an externally-located aspiration mechanism in combination with the internally located irrigation mechanism formed in accordance with principles of the present disclosure and that was previously described in association with FIGS. 1-12.

As familiar to those skilled in the art, the outer tubular member 18 and the inner tubular member 22 are formed from biocompatible metallic materials, such as stainless steel, titanium alloys, and the like. Accordingly, at least the outer tubular member 18 defines a generally rigid member.

Embodiments of the present disclosure facilitate surgery involving narrow access to treatment sites within a body. For example, with respect to sinus surgeries and other skull-related surgical procedures, a micro-burring instrument having a low cross-sectional profile, in accordance with principles of the present disclosure, provides a distinct advantage over currently-accepted techniques employing external irrigation tubes which increase the cross-sectional profile of the instrument and which increase the likelihood of the instrument getting caught during use.

Although the present disclosure has been described with reference to preferred embodiments, workers skilled in the art will recognize that changes can be made in form and detail without departing from the spirit and scope of the present disclosure.

What is claimed is:

1. A surgical instrument comprising:
an outer tubular member having a proximal section, an intermediate section, a distal section, and a side wall,
wherein the side wall includes an inner portion, an outer portion, at least one conduit between the inner portion and the outer portion that extends from the proximal section to the distal section,
wherein the inner portion of the side wall also defines a central lumen extending from the proximal section to the distal section, and
wherein the at least one conduit is open at an end of the distal section; and
an inner tubular member rotatably received within the central lumen, a distal end of the inner tubular member forming a bur extending distally beyond, and exposed relative to, the distal section of the at least one conduit to position the at least one conduit to direct a flow of fluid onto and near the bur.

2. The surgical instrument of claim 1, wherein the at least one conduit comprises a plurality of conduits uniformly spaced apart from each other about a circumference of the side wall.

3. The surgical instrument of claim 2, wherein the plurality of conduits comprise at least three conduits.

4. The surgical instrument of claim 1, comprising:
a hub including a lumen and a fluid port,
wherein the proximal section of the side wall comprises a proximal window and wherein the proximal section of the outer member is coaxially disposed within, and secured to, the lumen of the hub, and
wherein the fluid port of the hub is in communication with the at least one conduit via the proximal window.

5. The surgical instrument of claim 4, wherein the outer tubular member comprises:
a first sleeve defining the inner portion of the outer tubular member and including an inner surface and an outer surface, the inner surface of the first sleeve defining the central lumen, and
a second sleeve defining the outer portion of the outer tubular member and including an inner surface and an outer surface, the outer surface defining the proximal window.

6. The surgical instrument of claim 4, wherein the outer surface of first sleeve comprises:
a recess extending transverse to a longitudinal axis of the second sleeve at least partially about a circumference of the second sleeve and in communication with the proximal window of the second sleeve;
at least one elongate slot extending from the proximal section to the distal section and in fluid communication with the recess,
wherein the at least one slot and the inner surface of the second sleeve define the at least one conduit.

7. The surgical instrument of claim 6 wherein the at least one slot comprises a plurality of slots uniformly spaced apart from each other about the circumference of the inner surface of the outer member.

8. The surgical instrument of claim 6 wherein the at least one slot extends between and is defined by a pair of spaced apart raised protrusions formed on the outer surface of the first sleeve, wherein each protrusion is in sealing contact against the inner surface of the second sleeve, and wherein each protrusion extends from the proximal section to the distal section.

9. The surgical instrument of claim 6 wherein the proximal section of the outer surface of the first sleeve includes a non-recess portion sealingly secured to the inner surface of the second sleeve at a location proximal to the recess.

10. The surgical instrument of claim 4, comprising:
an irrigation fluid source in communication with the proximal window of the proximal section of the outer tubular member; and
a rotational controller configured to rotate the inner tubular member relative to the outer tubular member to cause rotation of the bur onto a target tissue at a surgical treatment site,
wherein the irrigation fluid is selectively directed to flow from the at least one conduit onto the surgical treatment site.

11. The surgical instrument of claim 10 wherein the bur defines a conduit including a distal opening and the inner tubular member defines a lumen in fluid communication with the conduit of the bur to define an aspiration pathway through an interior of the instrument.

12. The surgical instrument of claim 11 comprising a system including a negative pressure source in fluid communication with the aspiration pathway via a handpiece effectuating fluid communication to the lumen of the inner tubular member and the conduit of the bur.

13. A method of performing a burring procedure at a surgical treatment site, the method comprising:
providing an instrument including:
a generally tubular outer member including a proximal section, a distal section, and a side wall defining a central lumen extending from the proximal section to the distal section, wherein the side wall defines an interior passage including at least one conduit, and wherein the at least one conduit includes a distal opening and a proximal window; and
an inner tubular member rotatably received within the central lumen, wherein a distal end of the inner tubular member forms a bur extending distally beyond, and exposed relative to, the distal opening;
positioning a distal end of the instrument to place the bur in contact with a target tissue at the surgical treatment site;
rotating the burr to remove portions of the target tissue; and
supplying fluid from a fluid source external to the instrument, via the proximal window, through the at least one conduit and out of the distal opening onto the treatment site in association with operation of the bur.

14. The method of claim 13, comprising:
forming the bur to define a conduit including a distal opening and forming the inner tubular member to define a lumen;
arranging the conduit of the bur to be in fluid communication with the lumen of the inner tubular member;
providing an aspiration pathway from the distal opening through an interior of the instrument via the conduit of the bur and the lumen of the inner tubular member for connection to, and fluid communication with, a negative pressure source.

15. The method of claim 1, comprising;
forming the outer tubular member by:
providing an outer sleeve defining a lumen and a proximal window;
forming an inner sleeve to include an outer surface defining a plurality of elongate recesses extending along a majority of a length of the inner sleeve, wherein the recesses are generally uniformly spaced apart from each other about a circumference of the outer surface and wherein each recess is defined between a pair of elongate protrusions; and
inserting, and coaxially disposing, the inner sleeve within the lumen of the outer sleeve so that an inner surface of the outer sleeve and each respective recess defines a conduit and the conduits are in fluid communication with the proximal window.

16. The method of claim 15 wherein forming the inner tubular member includes:
forming a circular recess about the circumference of the outer surface of the inner sleeve proximal to the respective elongate recesses, wherein the circular recess is positioned to be in fluid communication with each respective conduit upon coaxially disposing the inner tubular member within the outer sleeve, and wherein the circular recess is in fluid communication with the proximal window of the outer sleeve; and
welding a proximal section of the inner sleeve proximal to the circular recess to the outer sleeve to sealingly secure the proximal section of the inner sleeve to the outer sleeve.

17. A surgical cutting instrument comprising:
an outer tubular member having a proximal section, an intermediate section, a distal section, and a side wall defining a central lumen extending from the proximal section to the distal section, including means for conducting fluid within a sidewall of the outer tubular member from the proximal section to the distal section and including a distal opening positioned to direct fluid at the bur and a treatment site; and
an inner tubular member rotatably received within the central lumen, a distal end of the inner tubular member forming a burr extending distally beyond, and exposed relative to, the distal section of the outer tubular member.

18. The surgical cutting instrument of claim 17 wherein the means for conducting comprises at least one conduit defined within the sidewall between an outer sleeve and an inner sleeve coaxially disposed within the inner sleeve, the inner sleeve being sealingly secured to the outer sleeve at the proximal section proximal to the at least one conduit.

19. The surgical cutting instrument of claim 18 wherein the at least one conduit comprises a plurality of conduits uniformly spaced apart about a circumference of the outer tubular member, and wherein each conduit is defined by an elongate recess formed in an outer surface of the inner sleeve and an inner surface of the outer sleeve secured over each of the respective recesses.
